(19) United States
(12) Patent Application Publication
(10) Pub. No.: US 2026/0026979 A1
(43) Pub. Date: Jan. 29, 2026

(54) ABSORBENT PAD WITH ADHESIVE FUNCTION

(71) Applicant: Jennifer Marie Nelson, Springville, UT (US)

(72) Inventor: Jennifer Marie Nelson, Springville, UT (US)

(21) Appl. No.: 18/787,785

(22) Filed: Jul. 29, 2024

(51) Int. Cl. A61F 13/472

(52) U.S. Cl. CPC A61F 13/472

(57) ABSTRACT

An absorbent pad with adhesive function operable to detachably affix to an interior surface of women's pants, the absorbent pad comprising a flexible polymeric fabric and absorbent material defining a plurality of slits.

### FIELD OF THE INVENTION

This invention relates to feminine hygiene, and more particularly relates to a disposable menstrual pad operable to detachably affix to an interior surface of pants.

### BACKGROUND

### Description of the Related Art

Feminine panty liners and absorbent liners operable to absorb discharge during a menstrual cycle and otherwise are known. These liners typically dispose in underwear or are separately strapped to the crotch area using thongs or compressive thongs.

There is an unmet need in the art for a liner which does not need to be included in separate clothing beneath pants. A panty liner with adhesive function which overcomes these difficulties inherent in the art is therefore desirable.

### SUMMARY

From the foregoing discussion, it should be apparent that a need exists for a panty liner with adhesive function. Accordingly, the present invention has been developed to provide a detachable absorbent pad, the absorbent pad comprising: an impermeable egg-shaped polymeric sheet between 70 and 90 mm wide, wherein the polymeric sheet covers a predetermined surface area; a secondary absorbent piece affixed superiorly to an obverse surface of the polymeric sheet, the secondary absorbent piece covering 60 to 80% of the predetermined surface area of the polymeric sheet; a thin polymeric film covering a reverse surface of the pad detachably affixed; an adhesive layering disposed between the thin polymeric film and the reverse surface, the adhesive layering operable to detachably affix to the pad to an interior surface of woman's pants.

In some embodiments, an exterior perimeter of the polymeric sheet defines a plurality of slits operable to form the pad over one of a concave and convex surface.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a top perspective view illustrating one embodiment of an absorbent pad panty liner with adhesive function in accordance with the present invention;

FIG. 2A is a lower perspective view illustrating one embodiment of an absorbent pad with adhesive function in accordance with the present invention;

FIG. 2B is a lower perspective view illustrating one embodiment of an absorbent pad with adhesive function in accordance with the present invention;

FIG. 3A is an environmental perspective view illustrating one embodiment of an absorbent pad with adhesive function in accordance with the present invention; and

FIG. 3B is an environmental perspective view of an interior cavity pants.

### DETAILED DESCRIPTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

It is an object of the present invention to provide an absorbent pad operable to detachably affix to an interior cavity of pants.

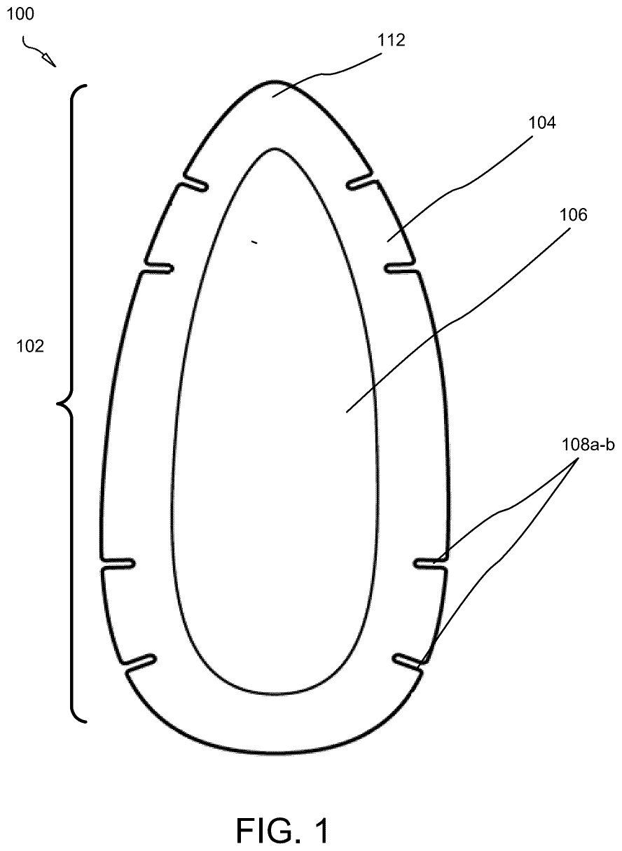

FIG. 1 is a top perspective view illustrating one embodiment of an absorbent pad with adhesive function 100 in accordance with the present invention.

The obverse surface 102 of the absorbent pad 100 is shown. The absorbent pad 100 comprises an ovoid, or egg-shaped, polymeric sheet 104 of between 0.05 mm and 2 mm in thickness. The polymeric sheet 104 may taper at a top surface to a point 112. The polymeric sheet may be impermeable to water.

A plurality of apertures, or slits 108a-b, dispose around a perimeter edge of the polymeric sheet 104. These slits 108 may be regularly or irregularly spaced around the perimeter and are operable to allow the polymeric sheet 104 to flex around a concave or convex surface defining an interior cavity of a pair of woman's pants. Affixed to the obverse surface 102 of the polymeric sheet 104 is a secondary piece 106 of fabric or absorbent material permanently adhered to the polymeric sheet 104. This secondary piece 106 may comprise polymeric or organic materials, such as paper, cork, or woven polymeric threads.

In various embodiments, the secondary piece 106 contours the shape of the outside edge of the polymeric sheet 104 without regard to the slits 108, but is smaller in shape. In various embodiments, the secondary piece 106 is 80% of the size of the polymeric sheet 102 as measured in surface area. The surface area of the polymeric sheet my consist or comprise a predetermined surface area. In various embodiments, the secondary piece 106 covers a surface area of between 50% and 90% of the surface area of the polymeric sheet 102.

In various embodiments, the polymeric sheet 104 is between 70 and 90 mm wide. The polymeric sheet 104 additionally or alternatively be between 120 and 180 mm tall. In some embodiments, the secondary piece 106 is between 40 and 60 mm wide and/or between 105 and 135 mm tall.

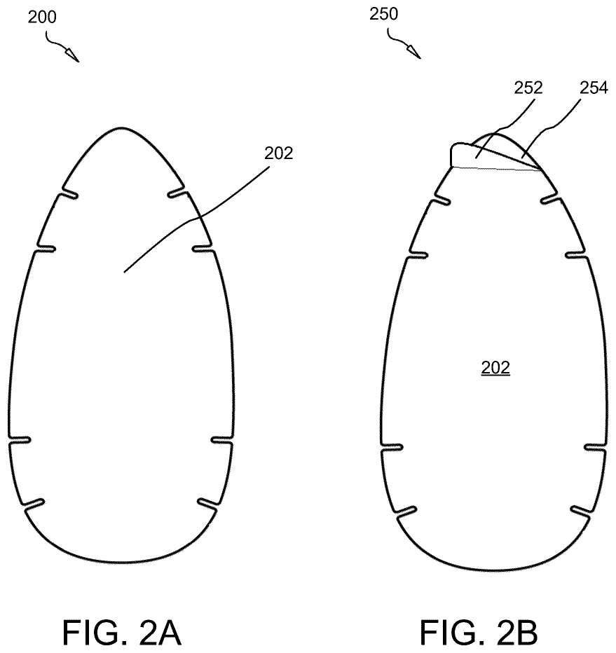

FIG. 2A is a lower perspective view illustrating one embodiment of an absorbent pad 200 with adhesive function in accordance with the present invention. The reverse surface of the absorbent pad 100, 200 is shown.

FIG. 2B is a lower perspective view illustrating one embodiment of an absorbent pad 250 with adhesive function in accordance with the present invention.

In some embodiments, a thin polymeric film 252 adheres to the reverse surface 202 of the pad 250. This flexible, thin polymeric film 252 covers an otherwise exposed adhesive covering a surface 254 of the pad 250 beneath the film 252. When the film 252 is removed, the surface 254 is exposed and the pad 250 places into a crotch area of women's pants.

The adhesive applied to the surface 254 may comprise a solvent-based adhesive or polymer-dispersion adhesive (i.e., emulsion adhesive), or natural or synthetic origin.

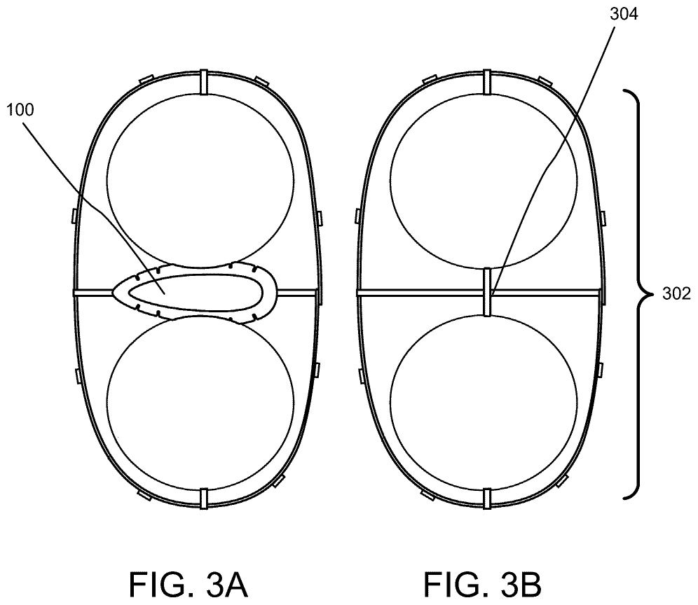

FIG. 3A is an environmental perspective view illustrating one embodiment of an absorbent pad with adhesive function 100 in accordance with the present invention.

The pad 100, further described above, positions between the legs of pants 302 in the crotch area 304. In this position, the pad 100 absorbs any vaginal discharge.

FIG. 3B is an environmental perspective view of an interior cavity pants 302 showing the area 304 when the pad 100 may be disposed. In other embodiments, the pad 100 is disposed in other positions on the pants, including in the legs or bottom area to absorb blood or discharge.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A detachable absorbent pad, the absorbent pad comprising:
an impermeable egg-shaped polymeric sheet between 70 and 90 mm wide, wherein the polymeric sheet covers a predetermined surface area;
a secondary absorbent piece affixed superiorly to an obverse surface of the polymeric sheet, the secondary absorbent piece covering 60 to 80% of the predetermined surface area of the polymeric sheet;
a thin polymeric film covering a reverse surface of the pad detachably affixed;
an adhesive layering disposed between the thin polymeric film and the reverse surface, the adhesive layering operable to detachably affix to the pad to an interior surface of woman's pants.

2. The absorbent pad of claim 1, wherein an exterior perimeter of the polymeric sheet defines a plurality of slits operable to form the pad over one of a concave and convex surface.
